(19) United States
(12) Patent Application Publication
(10) Pub. No.: US 2025/0082531 A1
(43) Pub. Date: Mar. 13, 2025

(54) EQUIPMENT FOR THE TRANSPORT OF BIOMEDICAL DEVICES

(71) Applicant: EUROSETS S.R.L., Medolla (MO) (IT)

(72) Inventors: Paolo FONTANILI, Medolla (MO) (IT); Antonio PETRALIA, Medolla (MO) (IT); Alessandro ZERBINI, Medolla (MO) (IT)

(73) Assignee: EUROSETS S.R.L., Medolla (MO) (IT)

(21) Appl. No.: 18/728,559

(22) PCT Filed: Feb. 20, 2023
(86) PCT No.: PCT/IB2023/051536

§ 371 Date: Jul. 12, 2024

(30) Foreign Application Priority Data
Feb. 24, 2022 (IT) 102022000003455

(51) Int. Cl. A61G 12/00; B62B 3/00; B62B 3/02; B62B 3/10

(52) U.S. Cl. CPC A61G 12/001; B62B 3/005; B62B 3/02; B62B 3/104

(57) ABSTRACT

Equipment for the transport of biomedical devices, comprises a load-bearing framing; movement device/apparatus/element/unit or the like of the framing with respect to a resting surface; at least one supporting plane associated with the framing and provided with removable anchoring device/apparatus/element/unit or the like of at least one biomedical device; at least one intermediate plane for the support of an additional device intended to be interconnected to the biomedical device, wherein the intermediate plane is associated movable by shifting with the framing along a direction of sliding between a working position, wherein it is positioned inferiorly to the supporting plane, and a maintenance position, wherein it is shifted with respect to the working position along the direction of sliding by arranging itself at least partly beyond the overall dimensions defined by the framing and wherein it is associated cantilevered with the framing.

### TECHNICAL FIELD

The present invention relates to a piece of equipment for the transport of biomedical devices.

### BACKGROUND ART

Equipment is known to transport, both in and out of the hospital, biomedical devices, such as extracorporeal blood circulation devices, e.g., oxygenators and centrifugal pumps, or other devices used in hospital practice and in emergency situations.

These known types of equipment are generally of the trolley type or the like, i.e., they comprise a load-bearing structure associated with means of movement, of the wheel type, and supporting a resting plane for one or more biomedical devices.

These known types of equipment do have some drawbacks.

Since they have to support a plurality of devices which must be interconnected in order to enable the proper operation of extracorporeal circulation, they in fact do not allow for easy and practical maintenance of the same.

In order to carry out the necessary cleaning and maintenance operations of the devices placed on the equipment of known type, operators must in fact move the devices themselves, removing the connections in place and locating them elsewhere so that they can conveniently work on them. This limitation is particularly clear in maintenance operations on the heat exchanger which is connected to the oxygenator and which must undergo regular cleaning operations to eliminate any bacteria that may lurk inside and which can be highly harmful to the patient undergoing extracorporeal circulation.

As can be easily guessed, the removal of the devices to be maintained from the relevant transport equipment is not easy and involves non-negligible downtime. Alternatively, operators can also carry out the necessary maintenance operations by keeping the devices in place on the relevant equipment.

This solution, too, has drawbacks however, since the space available for operators to work is particularly small so that access to the devices is particularly difficult and uneasy.

Still another drawback is the difficulty of conveniently and easily placing all the auxiliary devices necessary for the operation of extracorporeal circulation, since the transport equipment of known type has small overall dimensions constrained by hospital requirements.

### DESCRIPTION OF THE INVENTION

The main aim of the present invention is to devise a piece of equipment for the transport of biomedical devices which enables easy maintenance of the various devices being transported.

Within this aim, one object of the present invention is to simplify maintenance operations without the need to remove the devices from the relevant equipment. Another object of the present invention is to enable easy and convenient placement and connection of the various devices necessary for the operation of extracorporeal circulation.

Another object of the present invention is to devise a piece of equipment for the transport of biomedical devices which can overcome the aforementioned drawbacks of the prior art within the framework of a simple, rational, easy and effective to use as well as affordable solution.

The aforementioned objects are achieved by this piece of equipment for the transport of biomedical devices according to claim 1.

### BRIEF DESCRIPTION OF THE DRAWINGS

Other characteristics and advantages of the present invention will become more apparent from the description of a preferred, but not exclusive, embodiment of a piece of equipment for the transport of biomedical devices, illustrated by way of an indicative, yet non-limiting example, in the accompanying tables of drawings in which:

FIG. 1 is an axonometric view of a piece of equipment for the transport of biomedical devices according to the invention with the intermediate plane in the working position;

FIG. 2 is a view of the equipment in FIG. 1 with the intermediate plane in the maintenance position;

FIG. 3 is a sectional view of the equipment in FIG. 1;

FIG. 4 is a magnifying view of a detail of the equipment in FIG. 3;

FIG. 5 is a magnifying view of a detail of the equipment in FIG. 1, without the heat exchanger;

FIG. 6 is a partial exploded view of the equipment in FIG. 1;

FIG. 7 is an axonometric view of the equipment in FIG. 1 with the movable portion of the gripping element in the transport position.

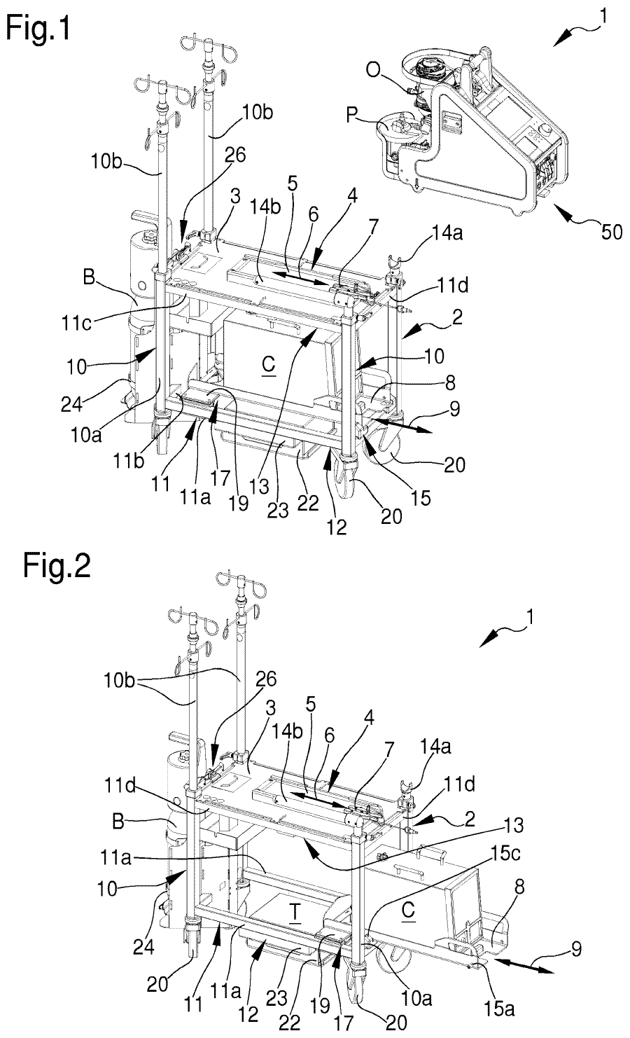

### EMBODIMENTS OF THE INVENTION

With particular reference to these figures, reference numeral 1 globally indicates a piece of equipment 1 for the transport of biomedical devices.

The equipment 1 comprises a load-bearing framing 2, movement means 20 of the framing 2 with respect to a resting surface S, at least one supporting plane 3 associated with the framing 2 and provided with removable anchoring means 4 of at least one biomedical device O, P.

More particularly, at least one piece of equipment for the support of biomedical devices 50 such as, e.g., an oxygenator O and the relevant pumping assembly P for extracorporeal blood circulation can be positioned on the supporting plane 3. Specifically, the anchoring means 4 comprise at least one guiding element 5 associated with the supporting plane 3 and developing along one direction of insertion/extraction 6 and removable retaining means 7 associated with the guiding element itself. The guiding element 5 is engageable by shifting along the direction of insertion/extraction 6 by an anchoring element (not shown in detail in the figures) which can be associated with the equipment for the support of biomedical devices 50 and the retaining means 7 are adapted to clamp the anchoring element itself along the direction of insertion/extraction 6 upon reaching a predefined anchoring position.

According to the invention, the equipment 1 also comprises an intermediate plane 8, for the support of an additional device, e.g. of the type of a heat exchanger C, intended to be connected to the biomedical device O, P. The intermediate plane 8 is associated movable by shifting with the framing 2 along a direction of sliding 9 between a working position, wherein it is positioned inferiorly to the supporting plane 3, and a maintenance position, wherein it is shifted with respect to the working position along the direction of sliding 9 so as to arrange itself at least partly beyond the overall dimensions defined by the framing itself. In the maintenance position, the intermediate plane 8 is associated cantilevered with the framing 2.

Preferably, the intermediate plane 8 is substantially vertically aligned with the supporting plane 3 in the working position and is vertically offset with respect to the supporting plane 3 in the maintenance position.

Appropriately, the intermediate plane 8 is associated with the framing 2 in the maintenance position.

More particularly, the intermediate plane 8 is constrained to the framing 2 in the maintenance position.

The intermediate plane 8, in the maintenance position, thus has an extremity portion associated with the framing 2 and the opposite extremity portion arranged outside of the framing 2 and disengaged therefrom.

When the intermediate plane 8 is brought to the maintenance position, it is then easily accessible by operators to carry out maintenance operations, such as sterilizing the heat exchanger C in order to eliminate any bacteria inside it. Once maintenance operations are completed, the intermediate plane 8 can be returned to the working position.

More particularly, the framing 2 comprises a plurality of load-bearing elements 10, 11 having an elongated shape and associated with each other, of which a plurality of vertical elements 10 and a plurality of horizontal elements 11 interconnected with each other.

In the particular embodiment shown in the figures, the framing 2 consists of at least four vertical elements 10 and a plurality of horizontal elements 11 locked together with the vertical elements 10. In this description, the terms “vertical” and “horizontal” refer to the arrangement taken by the elements under the conditions of normal use of the equipment 1, i.e., with the movement means 20 resting on the resting surface S, such as the ground.

Appropriately, at least two of the vertical elements 10 comprise a fixed body 10a and a movable body 10b telescopically fitted within the relevant fixed body 10a. The movable body 10b can then be moved in a sliding manner within the fixed body 10a so as to adjust its height with respect to the resting surface S. Preferably, as in the embodiment shown in the figures, all the vertical elements 10 of the framing 2 comprise a relevant fixed body 10a and a relevant movable body 10b.

The movement means 20 are, e.g., of the type of swivel wheels which are associated with the vertical elements 10.

Advantageously, the framing 2 comprises at least a first lattice 12 supporting the intermediate plane 8 and provided with a pair of first longitudinal elements 11a, which develop parallel to the direction of sliding 9, and with a pair of first transverse elements 11b, which develop transverse to the longitudinal elements 11a.

In more detail, the longitudinal elements 11a are arranged substantially (i.e., unless machining tolerances) perpendicular to the transverse elements 11b and show greater extension than them. It follows, therefore, that the first lattice 12 has quadrangular conformation, elongated in the direction of sliding 9.

The framing 2 therefore has elongated conformation and the term “longitudinal”, used in this description with reference to the horizontal elements 11, therefore identifies that these elements develop in the direction of the length of the framing itself.

As can be seen from the figures, the framing 2 also comprises a second lattice 13 supporting the supporting plane 3 and provided with a pair of second longitudinal elements 11c, which develop parallel to the direction of sliding 9 and with a pair of second transverse elements 11d, arranged transverse to the second longitudinal elements 11c.

In more detail, again the second longitudinal elements 11c are arranged substantially perpendicularly to the second transverse elements 11d.

In other words, the first longitudinal elements 11a and the second longitudinal elements 11c are arranged parallel to each other and the first transverse elements 11b and the second transverse elements 11d are arranged parallel to each other.

Advantageously, the equipment 1 comprises removable clamping means 15 of the intermediate plane 8 with respect to the framing 2 in the working position. More particularly, the clamping means 15 comprise at least one pair of clamping elements 15a locked together with the intermediate plane 8 and at least one pair of seats 15b associated with the framing 2, wherein the seats 15b are adapted to receive the clamping elements 15a in the working position of the intermediate plane 8, preventing the movement thereof along the direction of sliding 9. The seats 15b then define relevant abutment walls for the clamping elements 15a. The clamping elements 15a are liftable with respect to the seats 15b, so that they can be carried outside the seats themselves and then be moved towards the maintenance position.

In addition, the clamping means 15 also comprise a pair of ascent elements 15c adapted to guide the clamping elements 15a within the seats 15b upon completion of the displacement of the intermediate plane 8 from the maintenance position to the working position. In actual facts, the ascent elements 15c define respective inclined surfaces adapted to lift the intermediate plane 8 to allow it to enter the inside of the seats 15b.

Appropriately, the seats 15b are defined at one of the first transverse elements 11b, and the clamping elements 15a are arranged at one end of the intermediate plane 8.

Preferably, the intermediate plane 8 comprises sliding means 17 coupled to the framing 2 by shifting along the direction of sliding 9.

In more detail, the sliding means 17 comprise at least one pair of rectilinear guiding elements 18 associated with the first longitudinal elements 11a in a sliding manner.

As can be seen from FIG. 4, the guiding elements 18 are substantially C-shaped so as to operate in conjunction with the similar profile of the first longitudinal elements 11a and are connected to each other by means of a connecting plate 19. The guiding elements 18 are then fitted on the first longitudinal elements 11a at their inner side, that is, the side facing the other first longitudinal element 11a.

The guiding elements 18 are arranged at the point where the end of the intermediate plane 8 opposite the clamping elements 15a is located.

Appropriately, the sliding means 17, and in particular the guiding elements 18, are adapted to interact with the vertical elements 10 as a result of the shift of the intermediate plane 8 along the direction of sliding 9 towards the maintenance position. The interaction between the sliding means 17 and the vertical elements defines the end-of-stroke position of the intermediate plane 8 along the direction of sliding 9.

In the preferred embodiment shown in the figures, the intermediate plane 8 also comprises a housing compartment 21 of an auxiliary unit, such as of the type of a power supply unit, connectable to at least one of either the biomedical device O, P or the heat exchanger C.

Preferably, the equipment 1 comprises at least one containment body 22 open at the top, which is associated with the framing 2 and adapted to contain a voltage stabilizer T. More particularly, the intermediate plane 8 is arranged on top of the containment body 22 in the working position and is displaced with respect thereto in the maintenance position so as to allow the access from the outside to the containment body itself.

The result, therefore, is that when the intermediate plane 8 is brought to the maintenance position, the voltage stabilizer T is easily accessible from the outside.

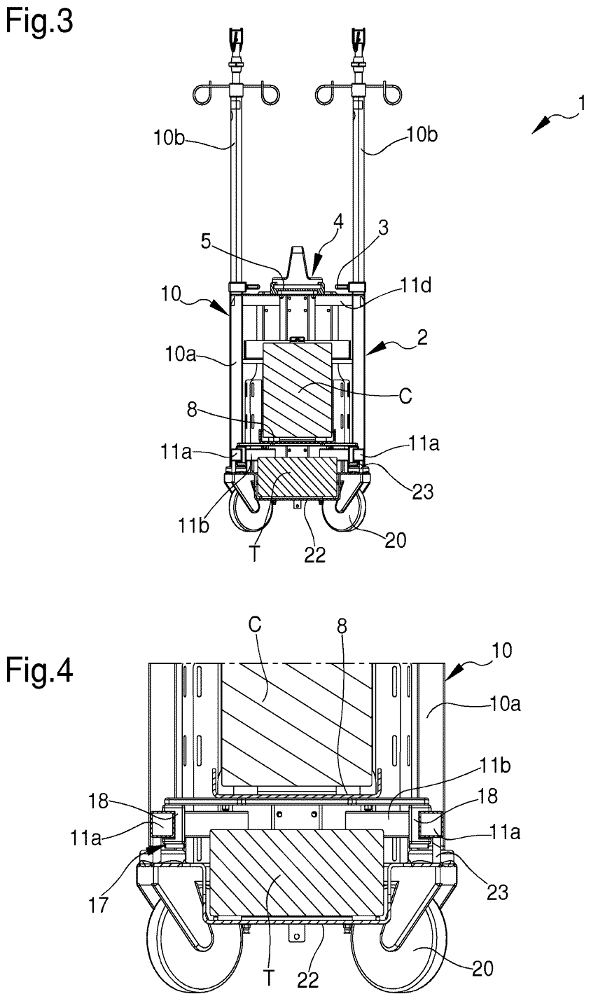

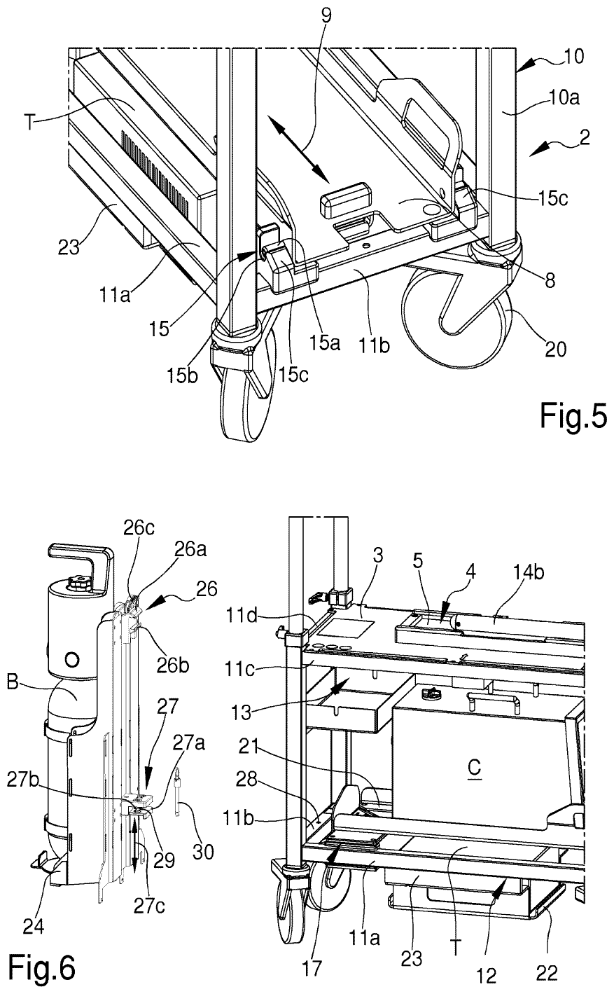

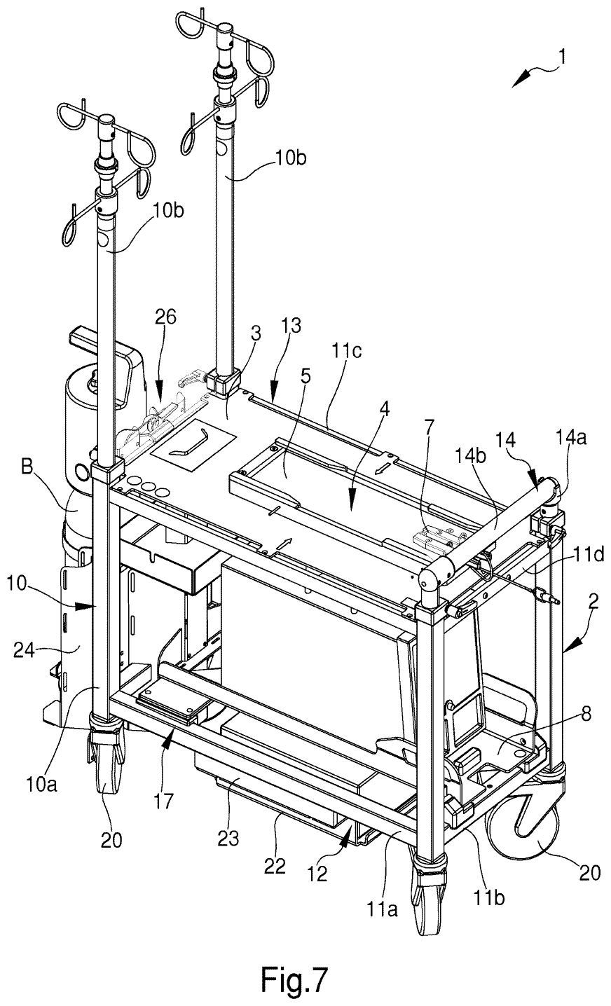

As can be seen from FIGS. 3 and 4, the containment body 22 is locked together with the first longitudinal elements 11a by means of relevant connecting walls 23 and the guiding elements 18 slide along the first longitudinal elements 11a within the space placed between the connecting walls 23. The connecting walls 23 are arranged, in use, substantially vertical.

Preferably, the equipment 1 comprises at least one supporting element 24 of at least one cylinder B containing an aeriform fluid, e.g., oxygen, associated with the framing 2 in a removable manner.

The supporting element 24 then defines a housing seat intended to receive a cylinder B and comprises removable coupling means 26, 27 to the framing 2. Advantageously, the coupling means 26, 27 comprise first coupling means 26 of the type of grippers adapted to engage, in a removable manner, with one of either the second longitudinal elements 11c or of the second transverse elements 11d.

In more detail, the first coupling means 26 comprise at least one upper gripping element 26a, a lower gripping element 26b, and control means 26c of the lower gripping element 26b operable to allow/prevent the displacement thereof with respect to the upper gripping element 26a. The control means 26c are, e.g., of the type of a lever movable between an adjustment position and a clamping position wherein it allows and prevents, respectively, the displacement of the lower gripping element 26b with respect to the upper gripping element 26a. Preferably, the coupling means 26, 27 also comprise second coupling means 27 adapted to operate in conjunction with the first lattice 12.

More particularly, the second coupling means 27 comprise at least one abutment element 27a adapted to lean on one of either the first longitudinal elements 11a or the first transverse elements 11b.

Appropriately, the second coupling means 27 comprise elastic means 27b, e.g. of the type of one or more springs, associated with the abutment element 27a to allow it to be displaced along a direction of adjustment 27c as a result of the interaction with one of the first elements 11a, 11b. The elastic means 27b thus allow the position of the abutment element 27a to be varied along the direction of adjustment 27c depending on the size of the first elements 11a, 11b with which it interacts. In other words, the abutment element 27a as a result of the interaction with the upper edge of one of the first elements 11a, 11b causes the elastic means 27b to compress or expand.

Advantageously, at least one of the first transverse elements 11b comprises a cavity 28 and the abutment element 27a comprises a through-hole 29 which can be positioned in alignment with the cavity 28. The second coupling means 27 then comprise at least one fastening element 30 insertable in a removable manner within the through-hole 29 and within the cavity 28 when aligned with each other. The fastening element 30 allows, therefore, the abutment element 27a to be solidly fastened to the relevant first transverse element 11b in a manner that prevents the accidental removal thereof and facilitates the transport thereof.

On the other hand, the first longitudinal elements 11a have no cavity for the housing of the fastening element 30.

Therefore, the supporting element 24 can only be solidly constrained to the first transverse elements 11b; this is because the positioning at the point where the longitudinal elements 11a, 11c are located would result in an unbalancing of the framing 2 and, therefore, would not allow safe movement of the equipment 1. The positioning of the supporting element 24 at the point where the longitudinal elements 11a, 11c are located is therefore permitted but only for the static use of the equipment 1, whereas when the latter is to be moved, positioning at where the transverse elements 11b, 11d are located and, in particular, anchoring to the first longitudinal elements 11a is required.

In the preferred embodiment shown in the Figures, the equipment 1 comprises at least one gripping element 14, associated with the framing 2 and arranged at the point where one of the second transverse elements 11d is located. More particularly, the gripping element 14 comprises a fixed portion 14a and a movable portion 14b associated with the framing 2 in a revolving manner and movable between a transport position, wherein it is directed parallel to the second transverse elements 11d and operates in conjunction with the fixed portion 14a, and a release position, wherein it is directed substantially parallel to the second longitudinal elements 11c and is disengaged from the fixed portion 14a. More particularly, the movable portion 14b and the fixed portion 14a are associated with the movable body 10b of a relevant vertical element 10, so that the height thereof can be varied as required. As can be seen from the figures, the fixed portion 14a defines a seat for housing the movable portion 14b in the transport position.

In the transport position, the movable portion 14b is thus arranged transversely to the direction of insertion/extraction 6, so as to hinder the movement of the equipment for the support of the biomedical devices 50.

It has in practice been ascertained that the described invention achieves the intended objects, and in particular the fact is emphasized that the equipment according to the invention allows for easy and practical maintenance of the transported devices, especially the heat exchanger, without the need to remove the latter from the equipment itself.

In fact, the possibility of moving the intermediate plane in a sliding manner allows easy access to the device placed thereon in the maintenance position.

At the same time, the displacement of the intermediate plane to the maintenance position also provides easy access to the containment body below.

Again, the supporting body of the oxygen cylinder can be easily displaced, thanks to the coupling means, to the most convenient position from time to time. At the same time, the supporting body of the oxygen cylinder can only be solidly fastened to the first transverse elements, since they are provided with the cavities for housing the fastening element, so that it can be safely moved together with the framing, so as to avoid the risk of tipping over during transport due to incorrect positioning where the longitudinal elements of the framing itself are located.

The invention claimed is:

1. An equipment for the transport of biomedical devices, the equipment comprising:
a load-bearing framing;
movement means of said framing with respect to a resting surface;
at least one supporting plane associated with said framing and provided with removable anchoring means of at least one biomedical device; and
at least one intermediate plane for the support of an additional device capable of being interconnected to said biomedical device, wherein
said intermediate plane is associated movable by shifting with said framing along a direction of sliding between a working position, wherein
the intermediate plane is positioned inferiorly to said supporting plane,
and a maintenance position, wherein
the intermediate plane is shifted with respect to the working position along said direction of sliding by arranging so as to be arranged at least partly beyond the overall dimensions defined by said framing, and
wherein, in the maintenance position, the intermediate plane is associated cantilevered with said framing.

2. The equipment according to claim 1, wherein said intermediate plane is substantially vertically aligned with said supporting plane in the working position and is vertically offset with respect thereto in the maintenance position.

3. The equipment according to claim 1, further comprising: removable clamping means of said intermediate plane to said framing in said working position.

4. The equipment according to claim 3, wherein said clamping means comprise at least one pair of clamping elements locked together with said intermediate plane and at least one pair of seats associated with said framing and adapted to receive said clamping elements in the working position, thus preventing displacement thereof along said direction of sliding, said clamping elements being able to be lifted up to exit said seats so as to allow the displacement of the intermediate plane towards said maintenance position.

5. The equipment according to claim 1, wherein said intermediate plane comprises sliding means coupled to said framing by shifting.

6. The equipment according to claim 5, wherein said framing comprises at least a first lattice supporting said intermediate plane and provided with a pair of first longitudinal elements, which develop parallel to said direction of sliding, and with a pair of first transverse elements, and said sliding means comprise at least one pair of rectilinear guiding elements associated with said first longitudinal elements in a sliding manner.

7. The equipment according to claim 1, wherein said intermediate plane associated with said framing in said maintenance position.

8. The equipment according to claim 7, wherein said intermediate plane is constrained to said framing in said maintenance position.

9. The equipment according to claim 6, wherein said framing comprises a plurality of load-bearing elements locked together with each other, of which a plurality of vertical elements and a plurality of horizontal elements associated with said vertical elements and said sliding means are adapted to interact with said vertical elements as a result of the shift of said intermediate plane along said direction of sliding towards the maintenance position, the interaction between said sliding means and said vertical elements defining the end-of-stroke position of said intermediate plane along said direction of sliding.

10. The equipment according to claim 1, wherein said intermediate plane comprises at least one housing compartment of an auxiliary unit connectable to at least one of either said biomedical device or said additional device.

11. The equipment according to claim 6, further comprising: at least one containment body open at a top, which is associated with said framing and adapted to contain a voltage stabilizer, wherein said intermediate plane is arranged on top of said containment body in the working position and is displaced with respect thereto in the maintenance position thus allowing the access from the outside to said containment body.

12. The equipment according to claim 11, wherein said containment body is locked together with said first longitudinal elements by means of relevant connecting walls and said guiding elements slide along said first longitudinal elements within the space placed between said connecting walls.

13. The equipment according to claim 1, further comprising: a supporting element of at least one cylinder containing an aeriform fluid associated with said framing in a removable manner.

14. The equipment according to claim 13, wherein said supporting element comprises removable coupling means to said framing.

15. The equipment according to claim 14, wherein said framing comprises at least one second lattice adapted to support said supporting plane and provided with a pair of second longitudinal elements and with a pair of second transverse elements, and by the fact that said coupling means comprise first coupling means of the type of grippers adapted to engage with one of either said second longitudinal elements or of said second transverse elements in a removable manner.

16. The equipment according to claim 15, wherein said coupling means comprise second coupling means adapted to operate in conjunction with said first lattice.

17. The equipment according to claim 16, wherein said second coupling means comprise at least one abutment element adapted to lean on one of either said first longitudinal elements or said first transverse elements.

18. The equipment according to claim 17, wherein said second coupling means comprise elastic means associated with said abutment element to allow the abutment element to be displaced along a direction of adjustment as a result of the interaction with one of said first elements.

19. The equipment according to claim 18, wherein at least one of said first transverse elements comprises a cavity, that said abutment element comprises a through-hole which can be positioned in alignment with said cavity and said second coupling means comprise at least one fastening element insertable in said through-hole and said cavity in a removable manner.

20. The equipment according to claim 19, wherein said first longitudinal elements have no cavity for the housing of said fastening element.

21. The equipment according to claim 15, further comprising: at least one gripping element, associated with said framing and arranged at the point where one of said second transverse elements is located.

22. The equipment according to claim 21, wherein said gripping element comprises a fixed portion and a movable portion associated with said framing in a revolving manner and movable between a transport position, wherein the movable portion is oriented parallel to said second transverse elements and operates in conjunction with said fixed portion and a release position, wherein the movable portion is oriented substantially parallel to said second longitudinal elements and is disengaged from said fixed portion.

23. The equipment according to claim 1, wherein said intermediate plane comprises at least one housing compartment of an auxiliary unit connectable to at least one of either said biomedical device or said additional device, and further comprising:
at least one containment body open at a top, associated with said framing and adapted to contain a voltage stabilizer, wherein said intermediate plane is arranged on top of said containment body in the working position and is displaced with respect thereto in the maintenance position so as to allow the access from the outside to said containment body; and
one supporting element of at least one cylinder containing an aeriform fluid, wherein said supporting element comprises removable coupling means to said framing.
